(19) United States
(12) Patent Application Publication
(10) Pub. No.: US 2011/0144596 A1
(43) Pub. Date: Jun. 16, 2011

(54) SPOUT AND LIQUID CONTAINER WITH SPOUT

(75) Inventors: Yoshihiko Kobashi, Hiroshima (JP); Takahiko Kunishige, Hiroshima (JP); Yoshinori Sato, Hiroshima (JP)

(73) Assignee: JMS CO., LTD., Hiroshima-shi, Hiroshima (JP)

(21) Appl. No.: 13/058,457

(22) PCT Filed: Sep. 4, 2009
(86) PCT No.: PCT/JP2009/065471

§ 371 Date: Feb. 10, 2011

(30) Foreign Application Priority Data
Sep. 12, 2008 (JP) 2008-234877
May 26, 2009 (JP) 2009-126748

(51) Int. Cl. A61M 5/00

(52) U.S. Cl. 604/262; 604/257

(57) ABSTRACT

A spout (4) that can be attached to a liquid container and through which contents with which the liquid container is filled are discharged, the spout including: a tubular portion (9) in which a through hole (12) is formed and from a leading end of which the content is discharged; and ribs (20) that partition the through hole (12). When the tubular portion (9) is seen from the leading end side, the through hole (12) is divided into at least three parts by the ribs (20). Thus, the strength of the ribs (20) is easily secured, which is advantageous in preventing breakage or deformation of the ribs (20), and also erroneous piercing with a connecting needle is reliably prevented.

### TECHNICAL FIELD

The present invention relates to a spout for allowing discharge of the contents with which a liquid container is filled and a liquid container with a spout using the same.

### BACKGROUND ART

Tube feeding is known as a treatment for those who cannot take in food orally. Tube feeding is a treatment for directly delivering nutrition into the body through a tube. For tube feeding, a nutrient solution bag, for example, that is filled with a nutrient solution is used, and the nutrient solution that has been supplied from the nutrient solution bag and has flowed through the tube is delivered into the body. A supply system in which a content in a bag is delivered into the body through a tube as described above is also used for infusion in which a drug solution is administered.

FIG. 26 shows a schematic configuration diagram of an example of a nutrient solution supply system that uses a conventional nutrient solution bag and an example of a drug solution supply system that uses a conventional drug solution bag. A bag main body 101 of a nutrient solution bag 100 is filled with a nutrient solution. A tube 105 provided with a connecting portion 104 at a leading end thereof is connected to the nutrient solution bag 100.

In the state shown in FIG. 26, a spout 102 is sealed with a liquid stopper cap 103. The spout 102 and the leading end of the tube 105 can be connected to each other by removing the liquid stopper cap 103.

On the other hand, a bag main body 111 of a drug solution bag 110 is filled with a drug solution. This drug solution has been collected from a drug solution bottle (not shown) via a tube (not shown) connected to a tube connector 112.

For administration of the drug solution into the body, a needle 114 is inserted into a needle port 113. Thus, the drug solution can be delivered into the body through a tube connected to the needle 114.

Here, the connecting portion 104 is designed so as to fit to the spout 102 of the nutrient solution bag 100. Therefore, the connecting portion 104 cannot be fitted to the needle port 113 of the drug solution bag 110, and erroneous connection of the connecting portion 104 to the drug solution bag 110 usually does not occur.

On the other hand, the liquid stopper cap 103 is formed of a soft material in order to facilitate attachment to and detachment from the spout 102. Thus, the liquid stopper cap 103 can be pierced with the needle 114. In this case, the needle 114 will penetrate through the liquid stopper cap 103 and reach the inside of the spout 102. If such erroneous piercing occurs, the nutrient material is administered from an infusion line that supplies the drug solution, and so there has been the possibility of a medical accident occurring.

In order to prevent such erroneous piercing, the below-listed Patent Document 1, for example, proposes a configuration in which a structure for preventing penetration of a connecting needle is provided in a portion corresponding to the spout 102. This structure for preventing penetration of a connecting needle is, for example, a helical member, an intermediate plate member, or a net member provided inside a tubular portion, and penetration of a connecting needle is prevented by a member such as these.

### CITATION LIST

### Patent Document

Patent Document 1: JP 2007-39121A

### DISCLOSURE OF INVENTION

### Problem to be Solved by the Invention

However, the penetration preventing structure using the helical member proposed in Patent Document 1 is not a structure that stops penetration at a leading end portion of the tubular portion, but rather is a structure that is based on the assumption that the connecting needle penetrates into the tubular portion to some extent. Moreover, the penetration preventing structure using the intermediate plate member or the net member is a structure in which deformation or cracking is likely to occur when the connecting needle is pushed M.

The present invention has been made to solve conventional problems such as those described above, and it is an object thereof to provide a spout that can more reliably prevent erroneous piercing with a connecting needle and a liquid container with the spout.

### Means for Solving Problem

In order to achieve the above-described object, a spout of the present invention is a spout that can be attached to a liquid container and through which the contents with which the liquid container is filled are discharged. The spout includes a tubular portion in which a through hole is formed and from a leading end of which the content is discharged; and ribs that partition the through hole, wherein when the tubular portion is seen from the leading end side, the through hole is divided into at least three parts by the ribs.

A liquid container according to the present invention with a spout includes the spout according to the present invention.

### Effects of the Invention

According to the present invention, erroneous piercing with a connecting needle can be prevented more reliably.

### BRIEF DESCRIPTION OF DRAWINGS

FIG. 1 is a schematic view of a nutrient solution bag 1 according to an embodiment of the present invention.

FIG. 2 is an exploded perspective view of a relevant part of the nutrient solution bag 1 shown in FIG. 1.

FIG. 3 is a cross-sectional view of a spout according to the embodiment of the present invention.

FIG. 4(a) is a diagram showing a state in which the spout 4 has been attached to a port portion 3, and FIG. 4(b) is a diagram showing a state in which a liquid stopper cap 5 has been attached to the spout 4 in FIG. 4(a).

FIG. 5 is a cross-sectional view taken in a vertical direction in FIG. 4(b).

FIG. 6 is a diagram showing an example of a state in which a tube for administration of a nutrient solution has been connected to a tubular portion 9 of the spout 4, in the embodiment of the present invention.

FIG. 7 is a perspective view showing the spout according to the embodiment of the present invention.

FIG. 8 is a cross-sectional view taken in the direction of the central axis of the spout 4 shown in FIG. 7.

FIG. 9 is a perspective view showing a situation in which a connecting needle 120 attempts to enter the inside of the tubular portion 9, in the embodiment of the present invention.

FIG. 10 is a cross-sectional view taken in the direction of the central axis of the tubular portion 9 in FIG. 9.

FIG. 11 is a perspective view showing a situation in which the connecting needle 120 attempts to enter the inside of a small hole 21, in the embodiment of the present invention.

FIG. 12 is a cross-sectional view taken along line AA in FIG. 11.

FIG. 13 is a perspective view showing still another example of the situation in which the connecting needle 120 attempts to enter the inside of the tubular portion 9, in the embodiment of the present invention.

FIG. 14 is a plan view showing another example of ribs 20 according to the embodiment of the present invention.

FIG. 15 is a plan view showing still another example of the ribs 20 according to the embodiment of the present invention.

FIG. 16 is a schematic view of a nutrient solution bottle 30 according to an embodiment of the present invention.

FIG. 17 is an exploded perspective view of a relevant part of the nutrient solution bottle 30 shown in FIG. 16.

FIG. 18 is an enlarged view of a tubular portion 41 according to the embodiment of the present invention.

FIG. 19 is a perspective view showing a state immediately before attachment of a spout 33 to a bottle main body 31, in the embodiment of the present invention.

FIG. 20 is a perspective view immediately before attachment of a connector 45 and a tube 46 to the spout 33, in the embodiment of the present invention.

FIG. 21 is a perspective view of a state in which connection of the connector 45 and the tube 46 to the spout 33 has been completed, in the embodiment of the present invention.

FIG. 22 is a perspective view showing a situation in which the connecting needle 120 attempts to enter the inside of the tubular portion 41, in the embodiment of the present invention.

FIG. 23 is a cross-sectional view taken in the direction of the central axis of the tubular portion 41 in FIG. 22.

FIG. 24 is a perspective view showing a situation in which the connecting needle 120 attempts to enter the inside of a small hole 44, in the embodiment of the present invention.

FIG. 25 is a cross-sectional view taken along line CC in FIG. 24.

FIG. 26 is a schematic configuration diagram showing an example of a nutrient solution supply system that uses a conventional nutrient solution bag and an example of a drug solution supply system that uses a conventional drug solution bag.

### DESCRIPTION OF THE INVENTION

According to the present invention, the through hole is partitioned into three or more parts by the ribs, and accordingly the strength of the ribs is easily secured, which is advantageous in preventing breakage or deformation of the ribs. Thus, erroneous piercing with a connecting needle also can be prevented more reliably.

In the spout of the present invention, it is preferable that the minimum distance from the position of the leading end of the tubular portion to the ribs increases toward the central axis of the tubular portion. With this configuration, the tip of the connecting needle abutting against a central portion of the ribs is unlikely to slip in a lateral direction, and thus the connecting needle is unlikely to go into the small holes demarcated by the ribs. Furthermore, the connecting needle abutting against a sloped portion is guided by the sloped portion and thus is likely to reach the central portion of the ribs.

Moreover, it is preferable that in a cross section of the ribs in the direction perpendicular to the central axis of the tubular portion, the ribs have a width that increases toward the central axis. With this configuration, it is possible to increase the area of the central portion of the ribs, and accordingly the connecting needle abutting against the central portion of the ribs is unlikely to go into the small holes demarcated by the ribs.

Moreover, it is preferable that when the ribs are seen from the leading end side of the tubular portion, a wide portion is formed in a portion where the ribs intersect each other, and the wide portion has a larger width than a portion of the ribs that connects the wide portion to the tubular portion. This configuration also makes it difficult for the connecting needle abutting against the central portion of the ribs to go into the small holes demarcated by the ribs.

Moreover, it is preferable that the spout further forms a step that extends out from an inner circumferential surface of the tubular portion toward the central axis of the tubular portion. With this configuration, even when a connecting needle having a small thickness has reached the small holes demarcated by the ribs, the distance to which the connecting needle enters can be suppressed.

Moreover, it is preferable that the spout further includes a base portion to be attached to the liquid container, wherein the tubular portion protrudes from the base portion, and a protruding dimension of the tubular portion from the base portion is 1 mm or more and 10 mm or less. This configuration is advantageous for transport or prevention of breakage of the tubular portion when the spout is attached to the liquid container, and also advantageous in securing the airtightness between the tubular portion and a tube attached to the tubular portion.

Moreover, it is preferable that the spout further includes an air-permeable filter. This configuration facilitates the flow of a liquid out of the liquid container.

Moreover, it is preferable that the liquid container is a pouch-like bag or a bottle.

It is preferable that the liquid container with a spout according to the present invention includes a liquid stopper cap that seals the spout, and when the liquid stopper cap is mounted to the spout, the distance between a leading end of the liquid stopper cap and the central portion of the ribs is 1 mm or less. With this configuration, the amount of a liquid that flows out in the case of erroneous piercing can be suppressed to a slight amount, and mounting of the liquid stopper cap also is facilitated.

Hereinafter, embodiments of the preset invention will be described with reference to the drawings.

### Embodiment 1

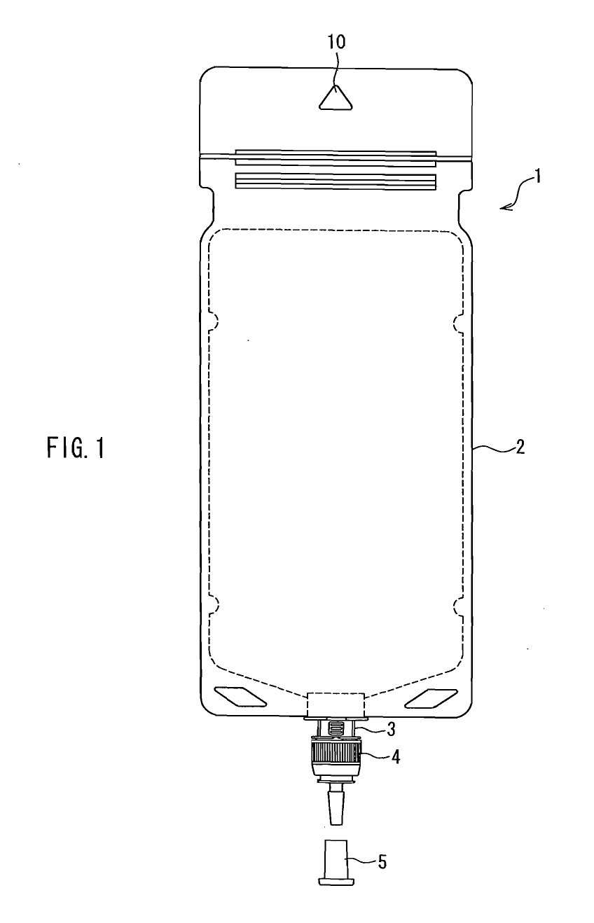

FIG. 1 shows a schematic view of a nutrient solution bag 1 according to Embodiment 1 of the present invention. A bag main body 2 serving as a liquid container is a portion to be filled with a nutrient solution. A spout 4 is attached to a port portion 3 that is integral with the bag main body 2. The spout 4 can be sealed by fitting a liquid stopper cap 5 thereto.

The bag main body 2 is formed by shaping a soft resin sheet into a pouch-like shape. The bag main body 2 can be formed by, for example, superposing two resin sheets on each other and joining peripheral edge portions together by heat welding or the like. An opening 10 for suspending the nutrient solution bag 1 is formed in one end of the bag main body 2.

Examples of the material for the resin sheet include polyethylene (PE), polypropylene (PP), polyethylene terephthalate (PET), polyvinyl chloride (PVC), polybutadiene, nylon, and ethylene-vinyl acetate copolymer (EVA). The resin sheet may be formed as a sheet having two or more layers, and the materials for the respective layers may be the same materials or may be different materials.

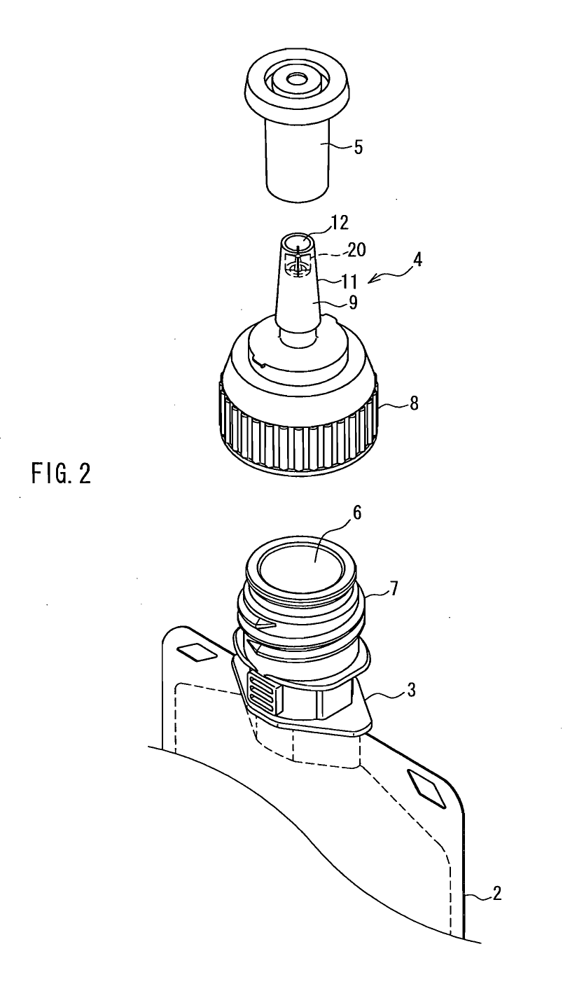

FIG. 2 is an exploded perspective view of a relevant part of the nutrient solution bag 1. The port portion 3 is a cylindrical member in which a through hole 6 is formed. The port portion 3 is integral with the bag main body 2, and a space that forms the through hole 6 is in communication with an inner space of the bag main body 2. The port portion 3 and the bag main body 2 can be joined together by, for example, performing heat welding in the state in which an end portion of the port portion 3 is sandwiched between two resin sheets. An external thread 7 to be screwed into the spout 4 is formed on an outer circumference of the port portion 3.

Examples of the material for the port portion 3 include polyethylene (PE), polypropylene (PP), polyacetal (POM), polyethylene terephthalate (PET), and polycarbonate (PC).

The spout 4 includes a cylinder portion 8 and a tubular portion 9 having a smaller diameter than the cylinder portion 8. The tubular portion 9 has a through hole 12 inside. A tapered surface 11 that increases in diameter from a leading end of the tubular portion 9 toward the cylinder portion 8 is formed on an outer circumferential surface of the tubular portion 9.

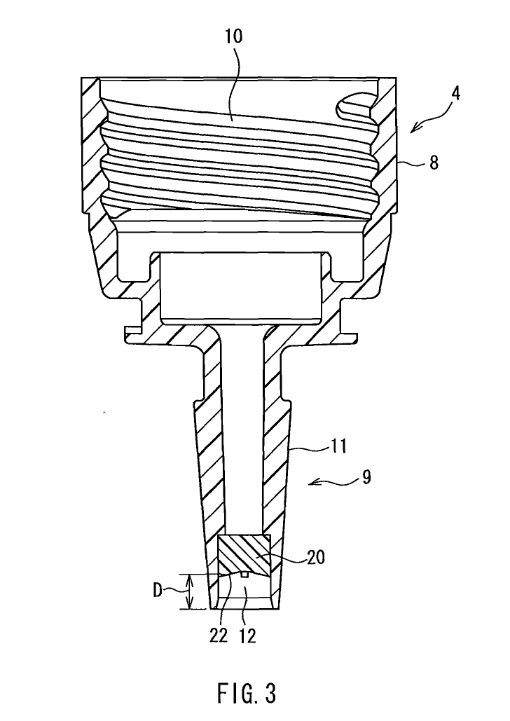

FIG. 3 shows a cross-sectional view of the spout 4. The spout 4 is a hollow member, and an inner space of the cylinder portion 8 is in communication with the through hole 12 inside the tubular portion 9. As shown in FIG. 3, an internal thread 10 is formed inside the cylinder portion 8. The spout 4 can be attached to the port portion 3 by screwing this internal thread 10 onto the external thread 7 (FIG. 2) of the port portion 3 by screwing. The same materials as those for the port portion 3 can be used as the material for the spout 4.

In FIG. 2, the liquid stopper cap 5 is a cylindrical member, and is opened on the spout 4 side and sealed on the opposite side. The liquid stopper cap 5 is for sealing the spout 4 and thereby preventing the contents from flowing out of the bag main body 2. The liquid stopper cap 5 is formed of a softer material than the spout 4 and can be attached to and detached from the tubular portion 9.

Examples of the material for the liquid stopper cap 5 include polypropylene (PP), polyethylene (PE), polycarbonate (PC), polybutadiene, polyvinyl chloride (PVC), ABS resin, and polyacetal (POM).

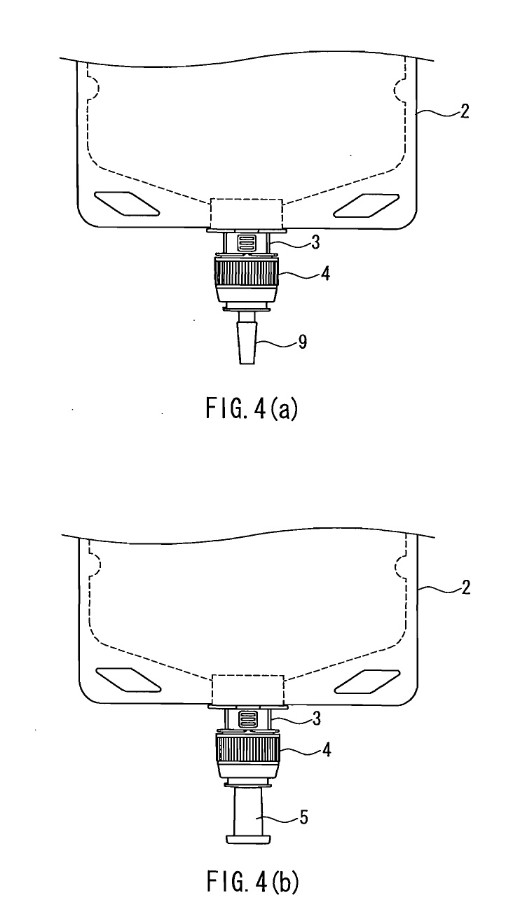

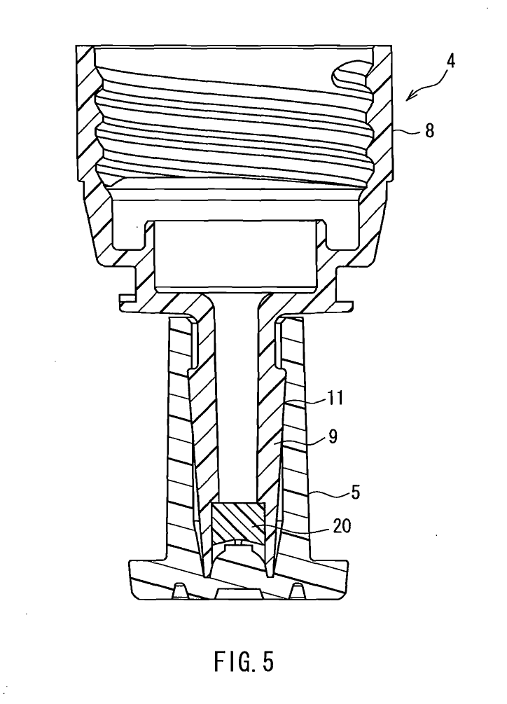

FIG. 4(a) shows a state in which the spout 4 has been attached to the port portion 3. FIG. 4(b) shows a state in which the liquid stopper cap 5 has been attached to the spout 4 in FIG. 4(a). FIG. 5 is a cross-sectional view taken in a vertical direction in FIG. 4(b). As shown in FIG. 5, the liquid stopper cap 5 is slid on so as to enclose the entire tubular portion 9 of the spout 4.

In the vicinity of an end portion of the tapered surface 11 on the cylinder portion 8 side, the outer diameter of the tapered surface 11 is larger than the inner diameter of the liquid stopper cap 5. Thus, in the state in which the liquid stopper cap 5 is mounted, the liquid stopper cap 5 partly comes into close contact with the tapered surface 11 due to elastic deformation of the liquid stopper cap 5, allowing the liquid stopper cap 5 to be mounted reliably.

The nutrient solution bag 1 is carried in the state in which the liquid stopper cap 5 is mounted as shown in FIGS. 4(b) and 5, and when the nutrient solution is to be administered, a tube for administration of the nutrient solution is connected to the tubular portion 9 of the spout 4 in the state in which the liquid stopper cap 5 is detached as shown in FIG. 4(a).

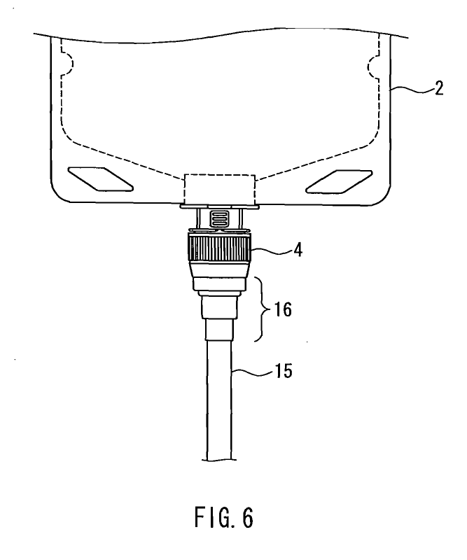

FIG. 6 shows an example of the state in which a tube for administration of the nutrient solution has been connected to the tubular portion 9 of the spout 4. In FIG. 6, the spout 4 and a tube 15 are connected to each other via a connecting portion 16 provided at a leading end of the tube 15. This connection is established by, for example, fitting a claw portion formed on the spout 4 into a recess formed in the connecting portion 16. In the state shown in FIG. 6, the nutrient solution in the bag main body 2 is administered into the body via the tube 15.

It should be noted that various types of connecting systems can be used for the connection between the spout 4 and the tube 15, and the connecting system in FIG. 6 is merely an example.

Hereinafter, a nutrient solution administration procedure that uses the nutrient solution bag 1 will be described more specifically. The nutrient solution bag 1 in an initial state before being filled with a nutrient solution is in the state in which the liquid stopper cap 5 is mounted to the spout 4 as shown in FIG. 4(b). From this state, the spout 4 is detached from the port portion 3 with the liquid stopper cap 5 still mounted. The detachment can be effected by rotating the spout 4 and thereby unscrewing it.

In the state in which the spout 4 has been detached from the port portion 3, the through hole 6 of the port portion 3 is exposed as shown in FIG. 2. The nutrient solution is injected into the bag main body 2 using this through hole 6 as an inlet.

After the injection of the nutrient solution into the bag main body 2, the spout 4 is again attached to the port portion 3 and thus returned to the state shown in FIG. 4(b). In this state, the nutrient solution bag 1 is carried. In this state, no tube is attached to the spout 4. Therefore, the nutrient solution bag 1 can be carried easily without being hindered by a long tube during carriage.

Moreover, the spout 4 is screwed onto the port portion 3, and the spout 4 is sealed with the liquid stopper cap 5. This prevents leakage of the content of the bag main body 2. Thus, the nutrient solution bag 1 also can be positioned in a horizontal orientation while being carried.

After the nutrient solution bag 1 has been carried, the liquid stopper cap 5 is detached as shown in FIG. 4(a), and in this state, the tube 15 for administration of the nutrient solution is connected to the tubular portion 9 of the spout 4 as shown in FIG. 6. At this time, the spout 4 is directed upward so as to prevent the nutrient solution from leaking. After the tube 15 has been connected, the bag is suspended by hooking the opening 10 (FIG. 1) onto a hook. Administration of the nutrient solution will be performed in this state.

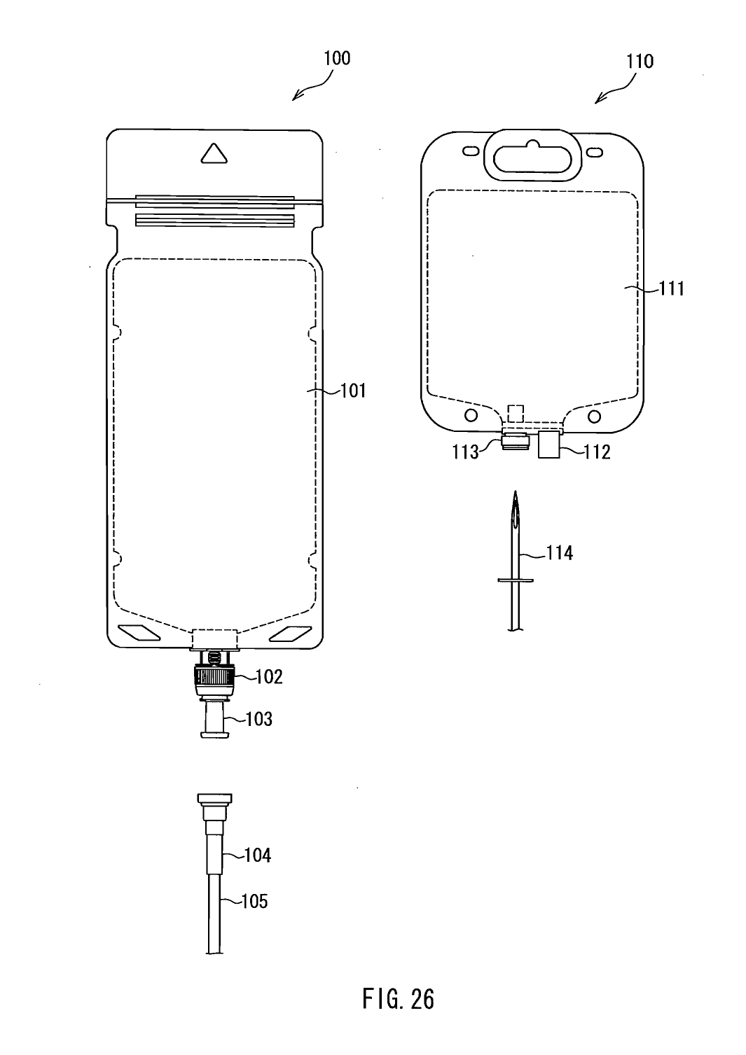

Here, as described above, FIG. 26 shows an example of the nutrient solution supply system that uses the conventional nutrient solution bag 100. FIG. 26 shows an example of a conventional combined use of both the nutrient solution bag 100 and the drug solution bag 110.

In this case, erroneous piercing may occur, in which the liquid stopper cap 103 of the nutrient solution bag 100 is pierced with the needle 114 that is to be inserted into the drug solution bag 110. The nutrient solution bag 1 according to the present embodiment has the same external configuration as that of the conventional nutrient solution bag 100 shown in FIG. 26.

Therefore, even when the nutrient solution bag 1 according to the present embodiment is used instead of the conventional nutrient solution bag 100 shown in FIG. 26, the liquid stopper cap 5 of the nutrient solution bag 1 shown in FIG. 4(b) may be pierced with the needle 114 that is to be inserted into the drug solution bag 110.

The spout 4 of the nutrient solution bag 1 according to the present embodiment has a structure that prevents such erroneous piercing. This will be described below.

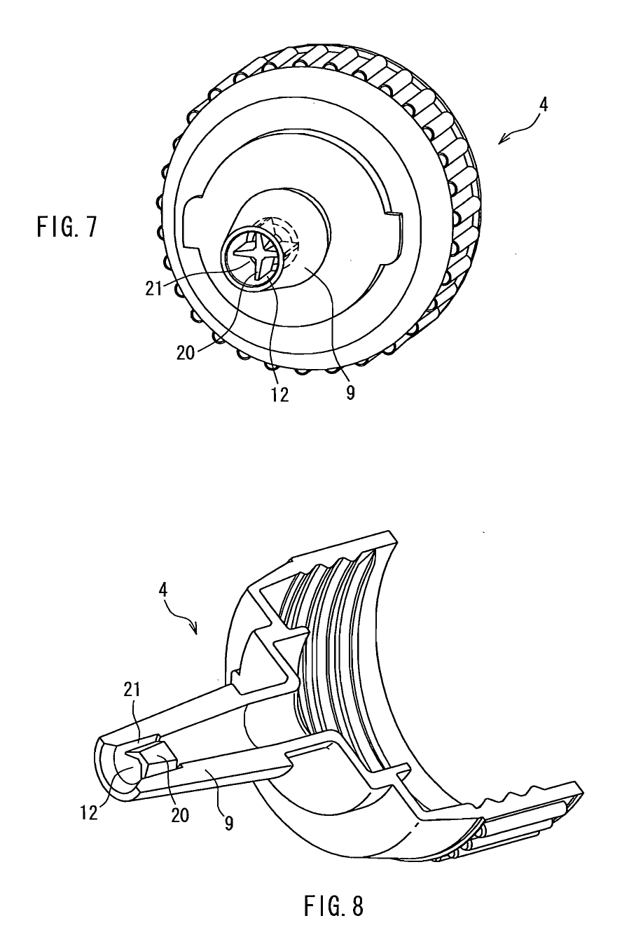

FIG. 7 shows a perspective view of the spout 4. FIG. 8 shows a cross-sectional view taken in the direction of the central axis of the spout 4 shown in FIG. 7. Ribs 20 that partition the through hole 12 are provided in the through hole 12 inside the tubular portion 9 of the spout 4. The through hole 12 is divided into four parts by the ribs 20. That is to say, the through hole 12 is divided into four small holes 21 in a portion where the ribs 20 are formed.

The spout 4 prevents erroneous piercing with a connecting needle by means of the ribs 20 formed in the through hole 12. In the state in which the tube 15 has been connected as shown in FIG. 6, there is no room for piercing with any connecting needle, and therefore erroneous piercing does not occur. In the state shown in FIGS. 4(b) and 5, the liquid stopper cap 5 made of a soft material may be pierced with a connecting needle for a bag that is different from the nutrient solution bag 1.

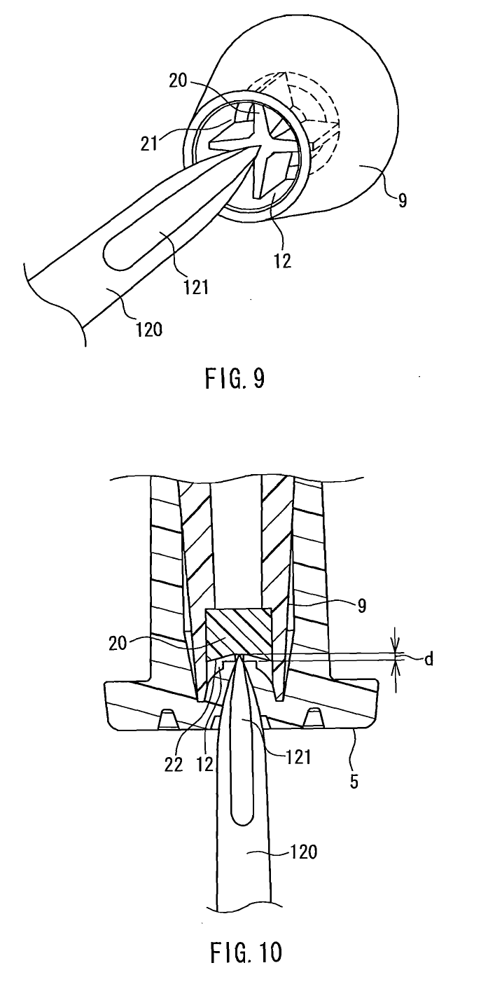

FIG. 9 is a perspective view showing the situation in which a connecting needle 120 attempts to enter the inside of the tubular portion 9. FIG. 10 shows a cross-sectional view taken in the direction of the central axis of the tubular portion 9 in FIG. 9. In FIGS. 9 and 10, the connecting needle 120 is a connecting needle for a bag that is different from the nutrient solution bag 1. The connecting needle 120 corresponds to the connecting needle 114 for the drug solution bag 110 in the example shown in FIG. 26.

As shown in FIG. 9, the tip of the connecting needle 120 abuts against a central portion of the ribs 20. In FIG. 9, although the liquid stopper cap 5 is omitted, the connecting needle 120 has penetrated through the liquid stopper cap 5 before reaching the central portion of the ribs 20 as shown in FIG. 10.

In the state shown in FIGS. 9 and 10, since the tip of the connecting needle 120 abuts against the central portion of the ribs 20, the connecting needle 120 cannot advance into the tubular portion 9 any further. That is to say, the distance to which the connecting needle 120 can pierce is limited to a slight distance, and the possibility that erroneous piercing will be noticed increases. In this case, the connecting needle 120 will be taken out, and thus erroneous piercing will be prevented.

Moreover, the length of a hole 121 in resin needles is usually long, and if the piercing distance of the connecting needle 120 is slight, the hole 121 is in a state of extending outside the liquid stopper cap 5 as shown in FIG. 10. In this state, even if the nutrient solution temporarily flows into the hole 121, the nutrient solution leaks out of the hole 121 outside the liquid stopper cap 5. In this case as well, erroneous piercing will be noticed and the connecting needle 120 will be taken out, and so erroneous piercing will be prevented.

On the other hand, the length of the hole 121 in metal needles is usually short (about 2 mm). Therefore, if the distance d between a leading end of the liquid stopper cap 5 and the central portion of the ribs 20 is set to a small distance, most of the hole 121 in the connecting needle 120 can be covered with the liquid stopper cap 5. Thus, even if the connecting needle 120 is left piercing, the flow of the content out of the bag main body can be suppressed. If the amount of the content that flows out can be suppressed to a slight amount, it also is possible to prevent the situation from going as far as administration of the content into the body.

More specifically, it is preferable that the distance d between the leading end of the liquid stopper cap 5 and the central portion of the ribs 20 is set to 1 mm or less. With this configuration, most of the hole 121 in the connecting needle 120 can be covered with the liquid stopper cap 5. Moreover, since this configuration is not a configuration in which the leading end of the liquid stopper cap 5 is brought into pressure contact with the ribs 20, mounting of the liquid stopper cap 5 also is facilitated.

Moreover, as shown in FIGS. 3 and 10, a side of the ribs 20 facing the leading end of the tubular portion 9 forms a sloped portion 22. The minimum distance D (FIG. 3) from the position of the leading end of the tubular portion 9 to the sloped portion 22 increases toward the central axis of the tubular portion 9. With this configuration, the tip of the connecting needle 120 abutting against the central portion of the ribs 20 as shown in FIGS. 9 and 10 is unlikely to slip in a lateral direction, and so the connecting needle 120 is unlikely to go into the small holes 21. Furthermore, the connecting needle 120 abutting against the sloped portion 22 is guided by the sloped portion 22 and is thus likely to reach the central portion of the ribs 20.

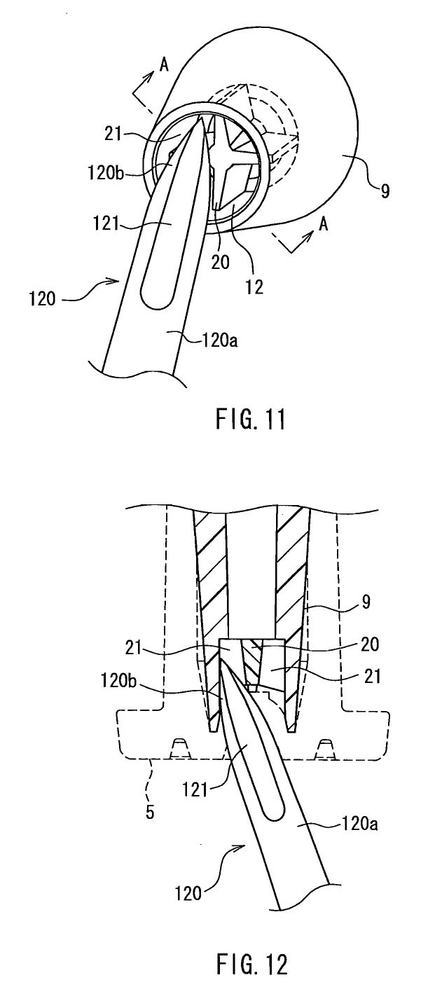

The example shown in FIGS. 9 and 10 is an example in which the tip of the connecting needle 120 has not reached the small holes 21, but there may be a case where the tip of the connecting needle 120 reaches a small hole 21. According to the present embodiment, even in the case where the tip of the connecting needle 120 has reached a small hole 21, the distance to which the connecting needle 120 enters can be suppressed. This will be described with reference to FIGS. 11 and 12.

FIG. 11 is a perspective view showing a situation in which the connecting needle 120 attempts to enter the inside of a small hole 21. FIG. 12 shows a cross-sectional view taken along line AA in FIG. 11. In FIG. 11, the connecting needle 120 has reached the small hole 21. In FIG. 11, although the liquid stopper cap 5 is omitted, the connecting needle 120 has penetrated through the liquid stopper cap 5 before reaching the small hole 21 as shown in FIG. 12.

The size of a tip portion 120b of the connecting needle 120 is smaller than that of a main body portion 120a. Therefore, if the size of the small holes 21 is set smaller than the size of the main body portion 120a, the entry of the connecting needle 120 is limited to the tip portion 120b. Thus, even in the case where the tip of the connecting needle 120 has reached a small hole 21, the distance to which the connecting needle 120 enters can be suppressed.

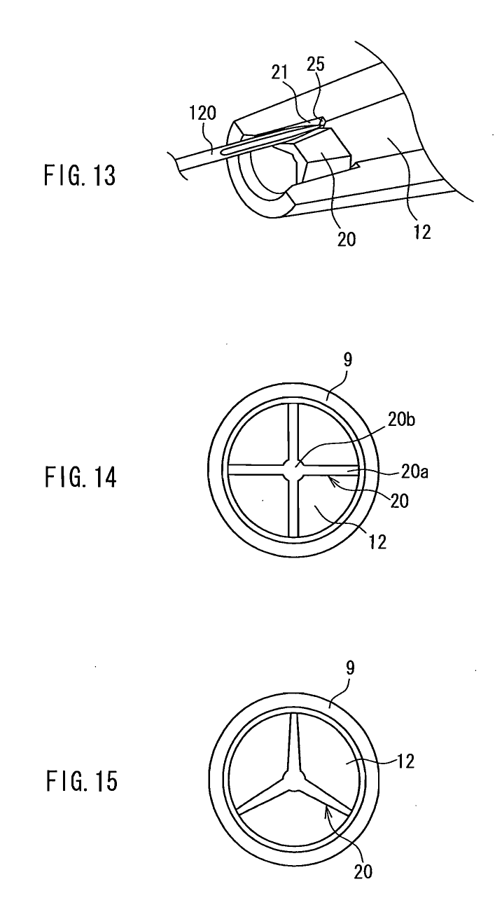

FIG. 13 is a perspective view showing still another example of the situation in which the connecting needle 120 attempts to enter the inside of the tubular portion 9. The illustration in FIG. 13 corresponds to a leading end portion of the tubular portion 9 in FIG. 8. In the example shown in FIG. 12, the size of the small holes 21 is set smaller than the size of the main body portion 120a of the connecting needle 120. In the example shown in FIG. 13, the distance to which the connecting needle 120 enters is suppressed even in the case where the connecting needle 120 whose main body portion 120a has a smaller size than the small holes 21 has reached a small hole 21.

As shown in FIG. 13, a step 25 is formed inside the tubular portion 9. The step 25 is provided farther inward than a leading edge of the ribs 20 and extends out from an inner circumferential surface of the tubular portion 9 toward the central axis of the tubular portion 9. In the example shown in FIG. 13, although the connecting needle 120 has penetrated into the small hole 21, the tip of the connecting needle 120 abuts against the step 25, and thus the entry of the connecting needle 120 is suppressed.

In the example shown in FIG. 13, the distance to which the connecting needle 120 enters is longer than that of the above-described examples shown in FIGS. 9 to 12.

However, it is possible to increase the possibility that erroneous piercing will be noticed by reducing the distance from a leading end surface of the tubular portion 9 to the step 25.

Moreover, if the hole 121 extends outside the liquid stopper cap 5 as shown in FIG. 10 when the connecting needle 120 has reached the step 25, erroneous piercing will be noticed due to leakage.

Next, in FIG. 9, for example, in a cross section of the ribs 20 taken in the direction perpendicular to the central axis of the tubular portion 9, the width of the ribs 20 increases toward the central axis of the through hole 12. With this configuration, the area of the central portion of the ribs 20 can be increased, and thus the connecting needle abutting against the central portion of the ribs 20 as shown in FIGS. 9 and 10 is unlikely to go into the small holes 21 demarcated by the ribs 20.

FIG. 14 is a plan view showing another example of the ribs 20. This drawing corresponds to a view of the ribs 20 as seen from the leading end side of the tubular portion 9. A wide portion 20b is formed in a portion where the ribs 20 intersect each other, and the width of the wide portion 20b is set larger than the width of a portion 20a of the ribs 20 that connects the wide portion 20b to the tubular portion 9. This configuration also makes it difficult for the connecting needle 120 abutting against the central portion of the ribs 20 as shown in FIGS. 9 and 10 to go into the small holes 21.

FIG. 15 is a plan view showing still another example of the ribs 20. Although the above-described embodiment is described using the example in which the through hole 12 is divided into four parts by the ribs 20, the through hole 12 may be divided into three parts by the ribs 20 as shown in FIG. 15. If the through hole 12 is divided into at least three parts by the ribs 20 as in this configuration, the strength of the ribs 20 can be secured easily, which is advantageous in preventing breakage or deformation, and erroneous piercing with the connecting needle can be more reliably prevented.

Moreover, a configuration in which the through hole is divided into five or more parts by the ribs 20 may also be employed. The larger the number of divisions, the more advantageous it is to the prevention of erroneous piercing and also to the prevention of breakage or deformation. On the other hand, the flow rate of the content that flows out through the spout 4 decreases. Therefore, the number of divisions made by the ribs 20 can be determined as appropriate within a range in which a necessary discharge amount can be secured.

It should be noted that although the spout 4 is attached to the bag main body 2 via the port portion 3 in the examples used in the foregoing description, the spout 4 may be directly attached to the bag main body 2.

### Embodiment 2

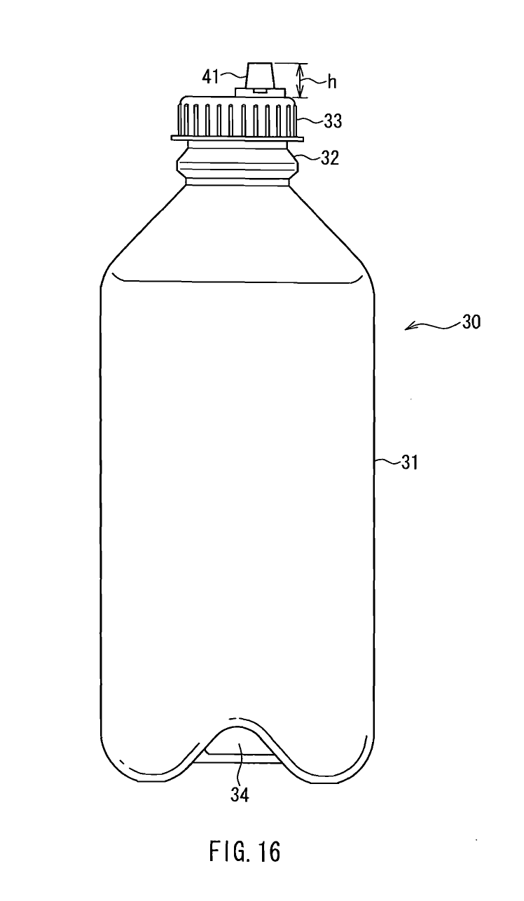

Hereinafter, Embodiment 2 of the present invention will be described. FIG. 16 shows a schematic view of a nutrient solution bottle 30 according to Embodiment 2 of the present invention. The nutrient solution bottle 30 includes a bottle main body 31, which is a hollow body serving as a liquid container, and a spout 33 attached to a mouth portion 32 thereof.

In Embodiment 1, the bag main body 2 to be filled with a nutrient solution is formed of a soft material, whereas in the present embodiment, the bottle main body 31 to be filled with a nutrient solution is formed of a hard material. Thus, the bottle main body 31 retains its outer shape unless an external force is particularly applied.

Moreover, Embodiment 1 is described using the example in which the bag main body 2 is not filled with a nutrient solution in the initial state, whereas the bottle main body 31 of the present embodiment is already filled with a nutrient solution in the initial state.

The bottle main body 31, which is a hollow body, is formed by, for example, blow molding a resin material. Examples of the resin material include polyethylene terephthalate (PET), polypropylene (PP), polyvinyl chloride (PVC), and nylon.

An opening 34 for suspending the nutrient solution bottle 30 is formed in a bottom portion of the bottle main body 31.

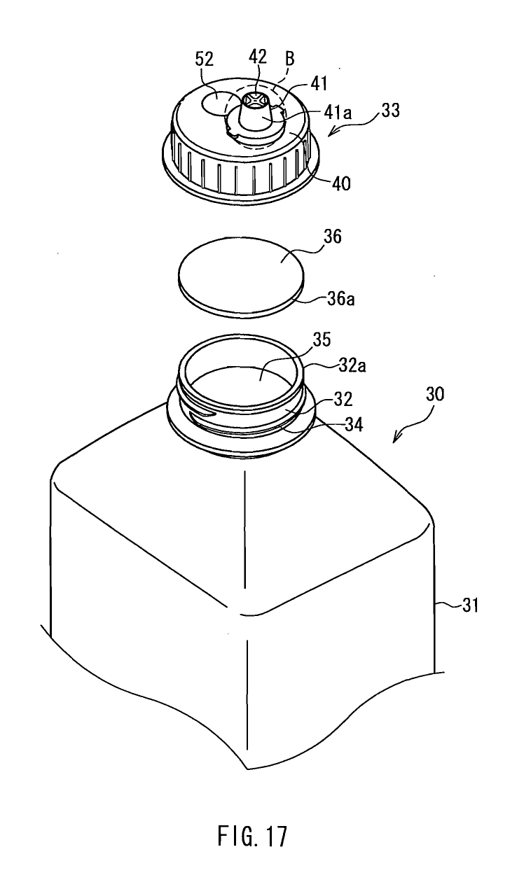

FIG. 17 is an exploded perspective view of a relevant part of the nutrient solution bottle 30. An external thread 34 to be screwed into the spout 33 is formed on an outer circumference of the mouth portion 32. The mouth portion 32 has a through hole 35 inside. A sealing member 36 is attached to the through hole 35, and thus an opening portion of the mouth portion 32 is hermetically sealed. The sealing member 36 is, for example, an aluminum foil, and a peripheral edge portion 36a thereof is bonded to a peripheral edge portion 32a of the mouth portion 32.

The spout 33 includes a base portion 40 and a tubular portion 41. The base portion 40 is a portion to be attached to the mouth portion 32 of the bottle main body 31. An internal thread (not shown) is formed inside the base portion 40. The spout 33 can be attached to the mouth portion 32 by screwing this internal thread onto the external thread 34 of the mouth portion 32.

The tubular portion 41 protrudes from the base portion 40. The tubular portion 41 has a through hole 42 inside. A tapered surface 41a that increases in diameter from a leading end of the tubular portion 41 toward the base portion 40 is formed on an outer circumferential surface of the tubular portion 41.

Examples of the material for the spout 33 include polyethylene (PE), polypropylene (PP), polyacetal (POM), polyethylene terephthalate (PET), and polycarbonate (PC).

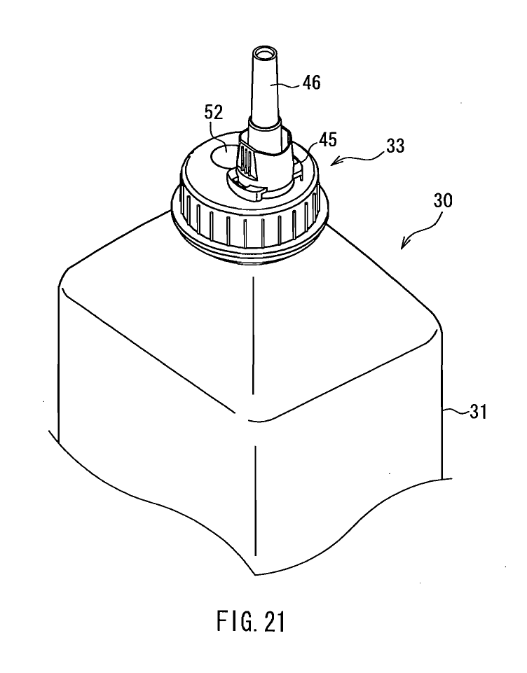

Moreover, a filter 52 is attached to the spout 33. Although details will be described later using FIG. 21, air is drawn into the bottle main body 31 through the filter 52 when the nutrient solution is caused to flow out through the tubular portion 41.

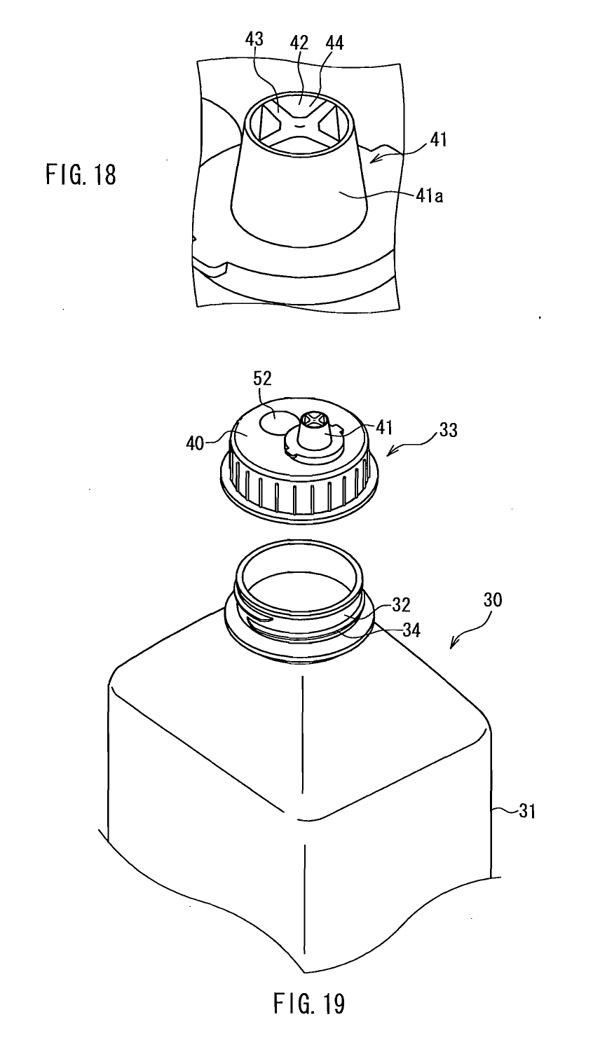

FIG. 18 shows an enlarged view of the tubular portion 41. This drawing corresponds to an enlarged view of a portion B in FIG. 17. Ribs 43 that partition the through hole 42 are provided in the through hole 42 inside the tubular portion 41. The through hole 42 is divided into four parts by the ribs 43. That is to say, the through hole 42 is divided into four small holes 44 in a portion where the ribs 43 are formed.

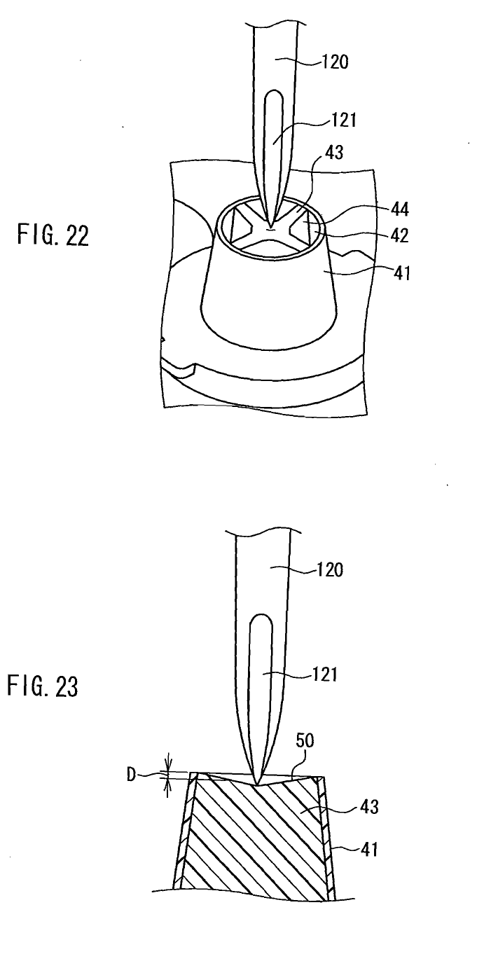

The tubular portion 41 prevents erroneous piercing with a connecting needle by means of the ribs 43 formed in the through hole 42. This will be described later with reference to FIGS. 22 to 25.

Hereinafter, a nutrient solution administration procedure that uses the nutrient solution bottle 30 will be specifically described. The nutrient solution bottle 30 is carried in the state shown in FIG. 16. In the state shown in FIG. 16, no tube is attached to the spout 33. Thus, the nutrient solution bottle 30 can be carried easily without being hindered by a long tube during carriage.

Moreover, the shorter the protruding dimension h (FIG. 16) of the tubular portion 41 from the base portion 40, the more advantageous it is for carrying the nutrient solution bottle 30 alone. Conversely, as the protruding dimension h becomes longer, the attachment of a tube to the tubular portion 41 becomes more difficult. Furthermore, the possibility of breakage due to fracture of the tubular portion 41 also increases. Therefore, the protruding dimension h is preferably 10 mm or less and more preferably 5 mm or less.

On the other hand, when the protruding dimension h excessively decreases, the area of contact between the tubular portion 41 and the tube decreases, and accordingly it becomes difficult to secure airtightness. Thus, the protruding dimension h is preferably 1 mm or more. For the foregoing reasons, the protruding dimension his preferably 1 mm or more and 10 or less and more preferably 1 mm or more and 5 mm or less.

Moreover, the mouth portion 32 of the bottle main body 31 is hermetically sealed with the sealing member 36 (FIG. 17), and leakage of the content of the bottle main body 31 thus is prevented. For this reason, the nutrient solution bottle 30 can also be positioned in a horizontal orientation while being carried.

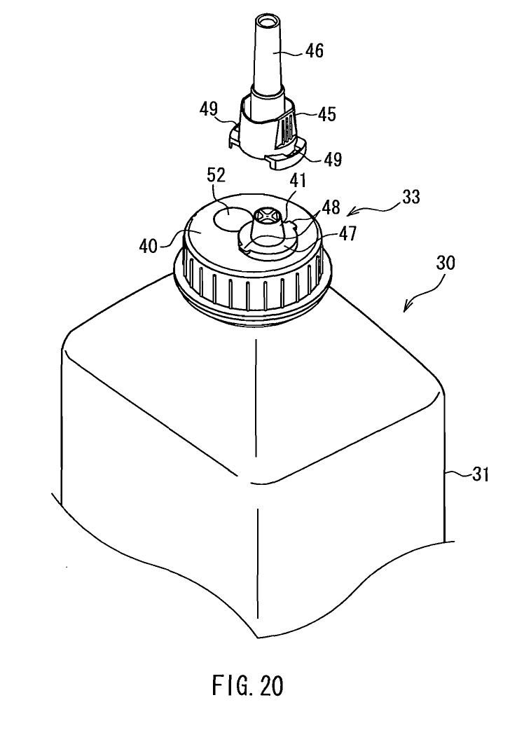

After the nutrient solution bottle 30 has been carried to an intended position, a connector 45 and a tube 46 (FIG. 20) are attached to the spout 33. During attachment of these, the spout 33 is detached temporarily from the bottle main body 31. The purpose of this is to partly or entirely remove the sealing member 36 with which the mouth portion 32 is hermetically sealed and thereby enable the nutrient solution in the bottle main body 31 to flow out.

FIG. 19 is a perspective view showing a state immediately before the attachment of the temporarily detached spout 33 to the bottle main body 31. In the state shown in this drawing, the sealing member 36 shown in FIG. 17 has been removed. The spout 33 is attached to the mouth portion 32 by screwing the internal thread (not shown) inside the base portion 40 of the spout 33 onto the external thread 34 of the mouth portion 32.

FIG. 20 shows a perspective view immediately before the attachment of the connector 45 and the tube 46 to the bottle main body 31 to which the spout 33 has been attached. The tube 46 for administration of the nutrient solution passes through the connector 45. Although the tube 46 has a predetermined length for the purpose of administration of the nutrient solution, only a part thereof on the connector 45 side is shown in FIGS. 20 and 21 for convenience of illustration. The tube 46 is formed of a softer material than the spout 33 and can be attached to and detached from the tubular portion 41.

The connector 45 and the tube 46 are connected to the spout 33 by press fitting the tubular portion 41 to an inner circumferential surface of the tube 46 and engaging the connector 45 with claw portions 48 provided on a pedestal portion 47. The connector 45 is engaged with the claw portions 48 by abutting the connector 45 against the surface of the base portion 40, rotating the connector 45, and thereby fitting the claw portions 48 into openings 49 formed in the connector 45. In this state, protrusions (not shown) formed on the connector 45 engage with an under side of the claw portions 48, so that the connector 45 is fixed to the spout 33, and thus unintentional disconnection of the connector 45 can be prevented.

Meanwhile, a flange portion (not shown) is formed at an end portion of the tube 46, and an end portion of the connector 45 engages with this flange portion. Thus, unintentional disconnection of the connector 45 from the tube 46 is prevented.

That is to say, in the state in which the connector 45 is fixed to the spout 33, the tube 46 is press fitted to the tubular portion 41 and also engages with the connector 45 fixed to the spout 33. This prevents unintentional disconnection of the tube 46.

It should be noted that various types of connecting systems can be used for the connection between the spout 33 and the tube 46, and the connecting system in FIG. 20 is merely an example.

FIG. 21 is a perspective view of a state in which the connection of the connector 45 and the tube 46 to the spout 33 has been completed. As described above, the sealing member 36 (FIG. 17) has been partly or entirely removed before the attachment of the spout 33. Thus, in the state shown in FIG. 21, an inner space of the bottle main body 31 is in communication with an inner space of the tube 46. Therefore, in the state in which the bottle main body 31 is suspended by hooking the opening 34 (FIG. 1) onto a hook, that is, in the state in which the bottle main body 31 in FIG. 21 is turned upside down, the nutrient solution in the bottle main body 31 can be caused to flow into the tube 46, and thus it is possible to administer the nutrient solution.

Here, during administration of the nutrient solution from a pouch-like bag formed of a soft material as in Embodiment 1, the bag deforms to a sheet-like state while the nutrient solution is flowing out. On the other hand, it is difficult for the bottle main body 31 formed of a hard material to deform like the pouch-like bag. An air-permeable filter 52 is attached to the spout 33 according to the present embodiment. The filter 52 is a hydrophobic filter and allows air to pass through, but not liquid.

When the nutrient solution is caused to flow out of the bottle main body 31, air is drawn into the bottle main body 31 through the filter 52 attached to the spout 33. This makes it easy for the nutrient solution to flow out of the bottle main body 31.

Next, in the case where the nutrient solution bottle 30 according to the present embodiment is used instead of the conventional nutrient solution bag 100 shown in FIG. 26, in the state in which the tube 46 has been connected to the spout 33 as shown in FIG. 21, there is no room for piercing with any connecting needle, and so erroneous piercing does not occur. On the other hand, in the state before the connection of the tube 46 as shown in FIG. 20, the tubular portion 41 may be pierced with the needle 114 that is to be inserted into the drug solution bag 110.

The spout 33 of the nutrient solution bottle 30 according to the present embodiment has a structure that prevents such erroneous piercing as is the case with Embodiment 1. This will be described below.

FIG. 22 is a perspective view showing a situation in which the connecting needle 120 attempts to enter the inside of the tubular portion 41. FIG. 23 shows a cross-sectional view taken in the direction of the central axis of the tubular portion 41 in FIG. 22. In FIGS. 22 and 23, the connecting needle 120 is not a needle for connection to the nutrient solution bottle 30. The connecting needle 120 corresponds to the connecting needle 114 for the drug solution bag 110 in the example shown in FIG. 26.

As shown in FIGS. 22 and 23, the tip of the connecting needle 120 abuts against a central portion of the ribs 43. In this state, the connecting needle 120 cannot advance into the tubular portion 41 any further. That is to say, the connecting needle 120 cannot pierce the tubular portion 41, and thus erroneous piercing is prevented.

Moreover, in the present embodiment, unlike Embodiment 1, a cap corresponding to the liquid stopper cap 5 (FIG. 10) is not attached to the tubular portion 41. Thus, it is possible to observe visually the leading end opening of the tubular portion 41 when piercing with the connecting needle 120. In this case, there is also the possibility that erroneous piercing will be prevented at a point in time when the ribs 43 are noticed.

Moreover, as shown in FIG. 23, a side of the ribs 43 facing the leading end of the tubular portion 41 forms a sloped portion 50. The minimum distance D from the position of the leading end of the tubular portion 41 to the sloped portion 50 increases toward the central axis of the tubular portion 41. With this configuration, the tip of the connecting needle 120 abutting against a central portion of the ribs 43 as shown in FIGS. 22 and 23 is unlikely to slip in a lateral direction, and thus the connecting needle 120 is unlikely to go into the small holes 44. Furthermore, the connecting needle 120 abutting against the sloped portion 50 is guided by the sloped portion 50 and is thus likely to reach the central portion of the ribs 43.

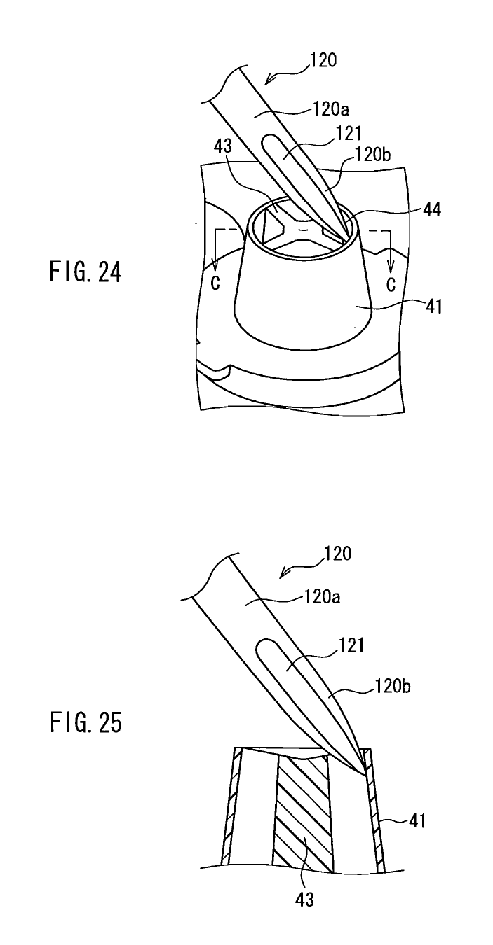

The example shown in FIGS. 22 and 23 is an example in which the tip of the connecting needle 120 has not reached the small holes 44. However, there may be a case where the tip of the connecting needle 120 reaches a small hole 44. According to the present embodiment, the distance to which the connecting needle 120 enters can be suppressed even in the case where the tip of the connecting needle 120 has reached a small hole 44. This will be described with reference to FIGS. 24 and 25.

FIG. 24 is a perspective view showing a situation in which the connecting needle 120 attempts to enter the inside of a small hole 44. FIG. 25 shows a cross-sectional view taken along line CC in FIG. 24. In FIG. 24, the connecting needle 120 has reached the small hole 44.

The size of the tip portion 120b of the connecting needle 120 is smaller than that of the main body portion 120a. Therefore, if the size of the small holes 44 is set smaller than the size of the main body portion 120a, the entry of the connecting needle 120 is limited to the tip portion 120b. Thus, the distance to which the connecting needle 120 enters can be suppressed even when the tip of the connecting needle 120 has reached a small hole 44.

Moreover, in FIG. 22, in a cross section of the ribs 43 taken in the direction perpendicular to the central axis of the tubular portion 41, the width of the ribs 43 increases toward the central axis of the tubular portion 41. This configuration is the same as the configuration shown in, for example, FIG. 9 of Embodiment 1, and the area of the central portion of the ribs 43 can be increased, so that the connecting needle abutting against the central portion of the ribs 43 as shown in FIGS. 22 and 23 is unlikely to go into the small holes 44 demarcated by the ribs 43.

Moreover, in the present embodiment as well, a configuration corresponding to the step 25 in FIG. 13 of Embodiment 1 may be provided. This makes it possible to suppress the distance to which the connecting needle 120 enters even in the case where the connecting needle 120 whose main body portion 120a has a smaller size than the small holes 21 has reached a small hole 44, as is the case with Embodiment 1. In this case, the possibility that erroneous piercing will be noticed can be increased by reducing the distance from the leading end surface of the tubular portion 9 to the step 25.

Moreover, in the present embodiment as well, a configuration corresponding to the wide portion 20b may be provided as is the case with the configuration in FIG. 14 of Embodiment 1, and a configuration in which the through hole is divided into three parts by the ribs may be employed as is the case with the configuration in FIG. 15. Furthermore, the present embodiment is also the same as Embodiment 1 in that a configuration in which the through hole is divided into five or more parts by the ribs may be employed.

It should be noted that although Embodiment 2 is described using the example in which the tubular portion 41 protrudes from the base portion 40 of the spout 33, the tubular portion 41 may be formed inside the spout 33 so that the tubular portion 41 does not protrude from the base portion 40.

Moreover, although Embodiment 2 is described using the example in which the connector 45 and the tube 46 that is made of a soft material are attached to the spout 33, it is sufficient that the nutrient solution can be administered from the spout 33, and an attachment portion may have other structures or follow other specifications.

For example, a specification in which the tube 46 is simply press fitted to the tubular portion 41 of the spout 33 without using the connector 45 may be followed.

Moreover, the liquid container is described using the pouch-like bag as an example in Embodiment 1 and using the hollow bottle as an example in Embodiment 2. However, it is possible to use a bottle in Embodiment 1 and to use a bag in Embodiment 2.

Moreover, since the spout according to Embodiment 1 or 2 can prevent erroneous piercing with a connecting needle as described above, the bag or the bottle to which the spout is attached is not limited to those for a nutrient solution and may be those used for other purposes.

### INDUSTRIAL APPLICABILITY

As described above, the spout according to the present invention more reliably can prevent erroneous piercing with a connecting needle and therefore is useful as a spout of for example, a nutrient solution bag.

### REFERENCE SIGNS LIST

1 nutrient solution bag
2 bag main body
4, 33 spout
5 liquid stopper cap
9, 41 tubular portion
11, 41a tapered surface
20, 43 rib
20a portion that connects wide portion to tubular portion
20b wide portion
21, 44 small hole
22, 50 sloped portion
25 step
30 nutrient solution bottle
31 bottle main body
40 base portion
52 filter

1. A spout that can be attached to a liquid container and through which contents with which the liquid container is filled are discharged, the spout comprising:
a tubular portion in which a through hole is formed and from a leading end of which the content is discharged; and
ribs that partition the through hole,
wherein when the tubular portion is seen from the leading end side, the through hole is divided into at least three parts by the ribs.

2. The spout according to claim 1, wherein a minimum distance from a position of the leading end of the tubular portion to the ribs increases toward a central axis of the tubular portion.

3. The spout according to claim 1, wherein in a cross section of the ribs taken in a direction perpendicular to a central axis of the tubular portion, the ribs have a width that increases toward the central axis.

4. The spout according to claim 1, wherein when the ribs are seen from the leading end side of the tubular portion, a wide portion is formed in a portion where the ribs intersect each other, and the wide portion has a larger width than a portion of the ribs that connects the wide portion to the tubular portion.

5. The spout according to claim 1, wherein the spout further forms a step that extends out from an inner circumferential surface of the tubular portion toward a central axis of the tubular portion.

6. The spout according to claim 1, further comprising a base portion to be attached to the liquid container, wherein the tubular portion protrudes from the base portion, and a protruding dimension of the tubular portion from the base portion is 1 mm or more and 10 mm or less.

7. The spout according to claim 1, further comprising an air-permeable filter.

8. The spout according to claim 1, wherein the liquid container is a pouch-like bag or a bottle.

9. A liquid container with a spout comprising the spout according to claim 1.

10. The liquid container with a spout according to claim 9, comprising a liquid stopper cap that seals the spout, wherein when the liquid stopper cap is mounted to the spout, a distance between a leading end of the liquid stopper cap and a central portion of the ribs is 1 mm or less.
